(19) United States
(12) Patent Application Publication
(10) Pub. No.: US 2026/0263070 A1
(43) Pub. Date: Sep. 10, 2026

(54) STAPLE CARTRIDGE WITH A KNIFE

(71) Applicant: Covidien LP, Minneapolis, MN (US)

(72) Inventors: Kenneth M. CAPPOLA, Monroe, CT (US); David M. FARASCIONI, Ridgefield, CT (US); Kenneth H. WHITFIELD, North Haven, CT (US)

(21) Appl. No.: 19/167,776

(22) PCT Filed: Mar. 22, 2024
(86) PCT No.: PCT/US2024/021070

§ 371 Date: Sep. 22, 2025

Related U.S. Application Data
(60) Provisional application No. 63/454,344, filed on Mar. 24, 2023.

(51) Int. Cl. A61B 17/072

(52) U.S. Cl. CPC A61B 17/07207; A61B 2017/07271; A61B 2017/07278; A61B 2017/07285

(57) ABSTRACT

A staple cartridge has a cartridge body with a central slot, an actuation sled, and a knife. The actuation sled includes a body having a base. The body has a guide member including a slot extending from the base. The actuation sled also includes angled camming wedges disposed on opposed sides of the guide member and a recess located in the base. A spring including a support and a finger is coupled to the actuation sled. The finger is resiliently connected to the support. The staple cartridge also includes a knife. The knife includes a foot, an arm, and a projection. The foot has a first pin disposed in the slot such that the knife is rotatable relative to the guide member. The projection engages the finger such that the finger biases the projection in a first direction.

### FIELD

This technology is generally related to surgical stapling devices, and more particularly, to surgical stapling devices including a staple cartridge with a knife.

### BACKGROUND

Surgical stapling devices configured for endoscopic use are well known and commonly used during surgical procedures to minimize patient trauma and reduce patient recovery times. Typically, endoscopic stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between open and clamped positions in response to movement of the drive assembly. The cartridge assembly includes a staple cartridge that supports staples that are ejected from the staple cartridge also in response to movement of the drive assembly.

Some stapling devices include a staple cartridge that can be replaced after each firing of the stapling device to facilitate reuse of the stapling device. Other stapling devices include a reload assembly that includes a staple cartridge and a drive assembly that can be replaced after each firing of the stapling device to facilitate reuse of the stapling device. The use of a reload assembly provides a new cutting blade for each firing of the stapling device to maintain the quality of cutting. The use of replaceable staple cartridges reduces the costs associated with a surgical procedure but generally requires reuse of the cutting blade. Repeated use of the same cutting blade may cause the cutting blade to become dull reducing the quality of cutting.

### SUMMARY

According to an aspect of the present disclosure, a staple cartridge includes a cartridge body, an actuation sled, and a knife. The cartridge body has a central slot extending along a length of the cartridge body. The actuation sled includes a body having a base. The body has a guide member including a slot. The guide member and slot extend from the base in an orientation that is transverse to the base. The body further includes angled camming wedges disposed on opposed sides of the guide member and a recess located in the base. The body further includes a spring having a support and a finger resiliently coupled to the support. The knife includes a foot, an arm, and a projection. The arm and the projection extend in opposite directions from the foot. The foot has a first pin disposed in the slot such that the pin is slidable in the slot and the knife is rotatable relative to the guide member. The arm includes a second pin and a knife blade. The second pin is engageable with a groove of a drive member. The projection engages the finger such that the finger biases the projection in a first direction thereby urging the arm in the first direction and defining a shielded position of the knife.

In an aspect of the present disclosure, translation of the drive member from a retracted position towards an advanced position may cause the second pin to slide from a distal position of the groove towards a proximal position of the groove thereby rotating the knife in a second direction opposite the first direction.

In another aspect of the present disclosure, translation of the drive member towards the advanced position may overcome the bias of the finger such that the knife rotates in the second direction towards a cutting position.

In a further aspect of the present disclosure, the drive member may include a nose that defines an acute angle with respect to a longitudinal axis of the staple cartridge and may be configured to urge tissue towards the knife blade.

In yet another aspect of the present disclosure, the cartridge body may further include staples disposed in a corresponding number of staple receiving pockets.

In an aspect of the present disclosure, the staple cartridge may further include pushers positioned within the staple receiving pockets and supporting the staples, and the angled camming wedges may be movable into engagement with the pushers to eject the staples from the staple receiving pockets.

In yet another aspect of the present disclosure, translation of the drive member from the advanced position towards the retracted position may cause the second pin to slide in the groove from the proximal position towards the distal position.

In an aspect of the present disclosure, translation of the drive member from the advanced position towards the retracted position may separate the actuation sled from the drive member and the finger may bias the projection in the first direction thereby rotating the knife in the first direction into the shielded position.

According to another aspect of the present disclosure, a surgical stapling instrument includes a handle, a shaft extending from the handle, a reload assembly, and a tool assembly. The reload assembly is coupled to the shaft and includes a drive member and a working member. The drive member is translatable between retracted and advanced positions and the working member is disposed at a distal end of the drive member. The tool assembly is coupled to the reload assembly. The tool assembly includes an anvil and a cartridge assembly that are pivotable with respect to each other between open and clamped configurations. The cartridge assembly includes a receptacle and a staple cartridge disposed in the receptacle. The staple cartridge has a cartridge body including a central slot, an actuation sled including a body and a base, and a guide member including an elongated slot. The guide member and the elongated slot extend transversely to the base. The staple cartridge further includes a spring having a support and a finger extending resiliently from the support. The support is disposed in a recess of the base. Additionally, the staple cartridge includes a knife having a foot, an arm, and a projection. The arm and projection extend in opposite directions from the foot. The foot includes a first pin disposed in the slot such that the knife is rotatable relative to the guide member. The arm includes a knife blade and a second pin. The second pin is engageable with a groove in the working member. The projection engages the finger such that the finger biases the projection in a first direction thereby urging the arm in the first direction and defining a shielded position of the knife. The knife is transitionable to a cutting position as the drive member translates from the retracted position towards the advanced position.

In an aspect of the present disclosure, translation of the drive member from the retracted position towards the advanced position may cause the second pin to slide from a distal position of the groove towards a proximal position of the groove thereby rotating the knife in a second direction opposite the first direction.

In another aspect of the present disclosure, translation of the drive member towards the advanced position may overcome the bias of the finger such that the knife rotates in the second direction towards an exposed position.

In aspects of the present disclosure, the working member may include a nose that defines an acute angle with respect to a longitudinal axis of the staple cartridge and may be configured to urge tissue towards the knife blade.

In a further aspect of the present disclosure, the cartridge body may further include staples disposed in a corresponding number of staple receiving pockets.

In yet another aspect of the present disclosure, the staple cartridge may include pushers positioned within the staple receiving pockets and supporting the staples, wherein the angled wedges may be movable into engagement with the pushers to eject the staples from the staple receiving pockets.

In an aspect of the present disclosure, translation of the drive member from the advanced position towards the retracted position may cause the second pin to slide in the groove from the proximal position towards the distal position.

In a further aspect of the present disclosure, translation of the drive member from the advanced position towards the retracted position may separate the actuation sled from the working member and the finger may bias the projection in the first direction thereby rotating the knife in the first direction into the shielded position.

According to a further aspect of the present disclosure, a tool assembly for use with a surgical stapling instrument includes an anvil assembly and a cartridge assembly where the anvil and cartridge assemblies are pivotable relative to each other between open and clamped positions. The cartridge assembly has a receptacle, a cartridge body with a central slot, and an actuation sled translatable in the cartridge body, and the actuation sled includes a base having a recess. The cartridge assembly includes a guide member extending from the base and the guide member includes an elongated slot that is transverse to the base. The guide member includes a spring coupled to the base, and the spring includes a resilient finger. The cartridge assembly also includes a knife having a foot, an arm, and a projection. The foot includes a first pin positioned in the elongated slot such that the knife is pivotable relative to the guide member. The arm includes a knife blade and a second pin. The second pin is slidably engageable with a groove of a drive member. The projection is operatively coupled with the resilient finger such that the resilient finger biases the projection in a first direction and defines a shielded position of the knife. The knife is transitionable to a cutting position as the drive member translates linearly through the cartridge body from a retracted position towards an advanced position wherein the second pin slides in the groove at an acute angle with respect to a longitudinal axis of the cartridge body.

In an aspect of the present disclosure, translation of the drive member from the retracted position towards the advanced position may cause the second pin to slide from a distal position of the groove towards a proximal position of the groove thereby rotating the knife in a second direction opposite the first direction.

In another aspect of the present disclosure, the drive member may include a nose that defines an acute angle with respect to the longitudinal axis and may be configured to urge tissue towards the knife blade.

In a further aspect of the present disclosure, translation of the drive member from the advanced position towards the retracted position may cause the second pin to slide in the groove from the proximal position towards the distal position and may separate the actuation sled from the drive member. The finger may bias the projection in the first direction thereby rotating the knife in the first direction into the shielded position.

Other features of the disclosure will be appreciated from the following description.

### BRIEF DESCRIPTION OF THE DRAWINGS

Various aspects of the disclosed staple cartridge and surgical stapling device are described herein below with reference to the drawings, wherein:

FIG. 1 is a perspective view of a surgical stapling instrument according to an aspect of the present disclosure;

FIG. 2 is a perspective view of a tool assembly of the surgical stapling instrument of FIG. 1 illustrating a staple cartridge separated from a jaw of the tool assembly;

FIG. 3 is an exploded perspective view, with parts separated, of the staple cartridge of FIG. 2 showing an actuation sled, staples, and staple pushers;

FIG. 4 is an enlarged view of the area of detail in FIG. 3 illustrating the actuation sled;

FIG. 5 is a bottom perspective view of the actuation sled of FIG. 4;

FIG. 6 is an exploded front perspective view, with parts separated, of the actuation sled of FIG. 5 illustrating a knife and a spring;

FIG. 7 is a top perspective view of the actuation sled of FIG. 6 without the knife or the spring;

FIG. 8 is a bottom cross-sectional view of the actuation sled of FIG. 5 taken along section line 8-8;

FIG. 9 is a bottom perspective view of another aspect of an actuation sled and a spring;

FIG. 10 is a front perspective view of a drive member of the surgical stapling instrument of FIG. 1;

FIG. 11 is a front perspective view of a drive member of the surgical stapling instrument of FIG. 1 illustrating a groove on a working member of the drive member;

FIG. 12 is a top cut-away view of a proximal portion of the tool assembly;

FIG. 13 is a side cross-sectional view of the staple cartridge of FIG. 2 taken along section line 13-13;

FIG. 14 is an enlarged view of the area of detail in FIG. 13;

FIG. 15 is a side cross-sectional view of the tool assembly of FIG. 2 taken along section line 15-15;

FIG. 16 is a side cross-sectional view of the tool assembly in an approximated configuration with the staple cartridge installed and the drive member in a retracted position;

FIG. 17 is an enlarged view of the area of detail in FIG. 16;

FIG. 18 is a side cross-sectional view of the tool assembly of FIG. 16 with the drive member between the retracted position and an advanced position;

FIG. 19 is an enlarged view of the area of detail in FIG. 18;

FIG. 20 is a side cross-sectional view of the tool assembly of FIG. 16 as the drive member and the knife translate distally towards the advanced position of the drive member;

FIG. 21 is a side cross-sectional view of the tool assembly of FIG. 16 showing the drive member as it translates from the advanced position towards the retracted position; and

FIG. 22 is a side cross-sectional view of the tool assembly of FIG. 21 showing the drive member translating proximally and separating from the actuation sled.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes a cartridge assembly having a replaceable staple cartridge that includes a knife and an actuation sled. The knife is pivotably coupled to the actuation sled and includes a knife blade that is movable from a shielded position to a cutting position in response to advancement of a drive member of the surgical stapling device. The knife and the actuation sled form part of the staple cartridge and are replaced after each firing of the surgical stapling device to provide a staple cartridge with a knife having a sharp edge for each firing of the surgical stapling device.

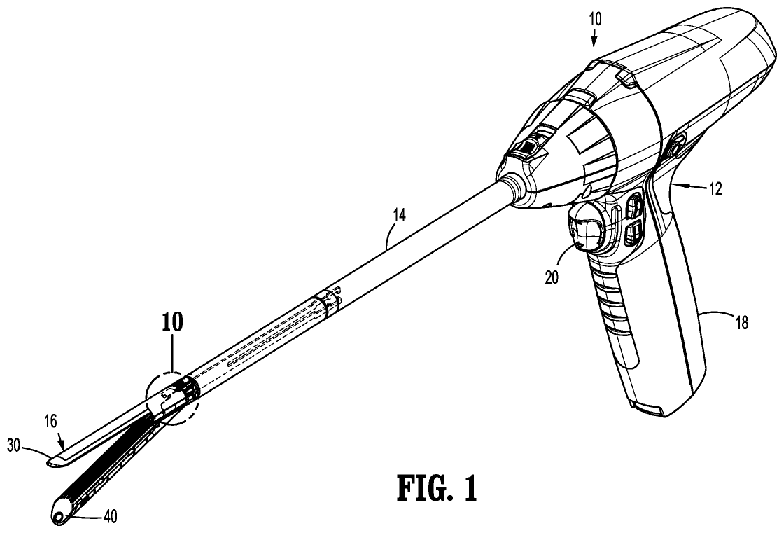

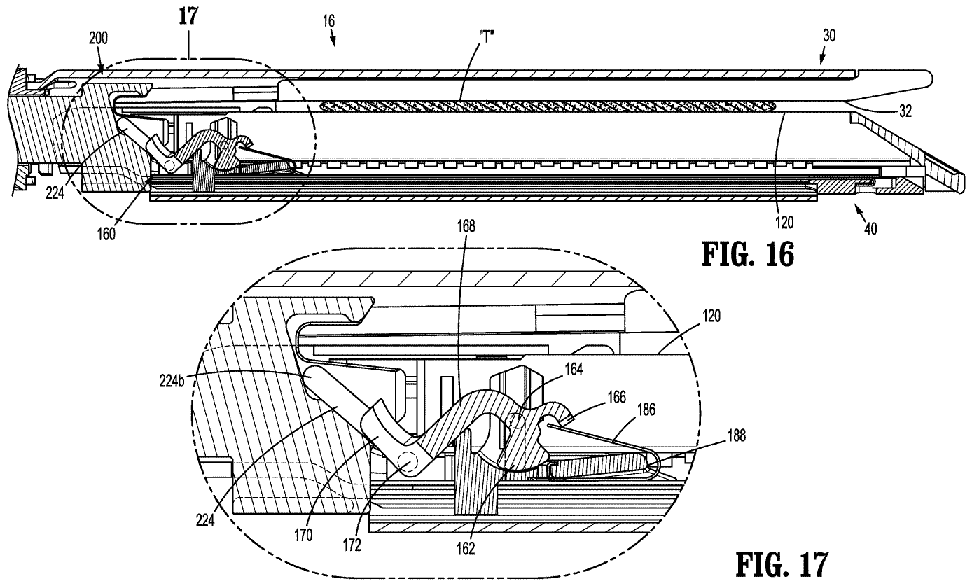

FIG. 1 illustrates a surgical stapling device shown generally as stapling device 10 that includes a handle assembly 12, a shaft 14 extending from the handle assembly 12, and a tool assembly 16. The handle assembly 12 is powered and includes a stationary handgrip 18 and actuation buttons 20. The actuation buttons 20 are operable to actuate various functions of the tool assembly 16 via the shaft 14, i.e., approximation of the tool assembly 16, and stapling and cutting of tissue “T” (FIG. 16). In certain aspects of the disclosure, the handle assembly 12 supports batteries (not shown) that provide power to the handle assembly 12 to operate the stapling device 10. Although the stapling device 10 is illustrated as a powered stapling device, it is envisioned that the advantages of this disclosure are suitable for use with manually powered stapling devices as well as robotically controlled stapling devices. U.S. Pat. No. 9,055,943 discloses a stapling device including a powered handle assembly and U.S. Pat. Nos. 5,865,361 and 6,241,139 disclose stapling devices with manually actuated handle assemblies.

The shaft 14 defines a longitudinal axis “X” and includes a proximal portion 14a and a distal portion 14b. The proximal portion 14a of the shaft 14 is coupled to the handle assembly 12. The distal portion 14b of the shaft 14 is coupled to the tool assembly 16.

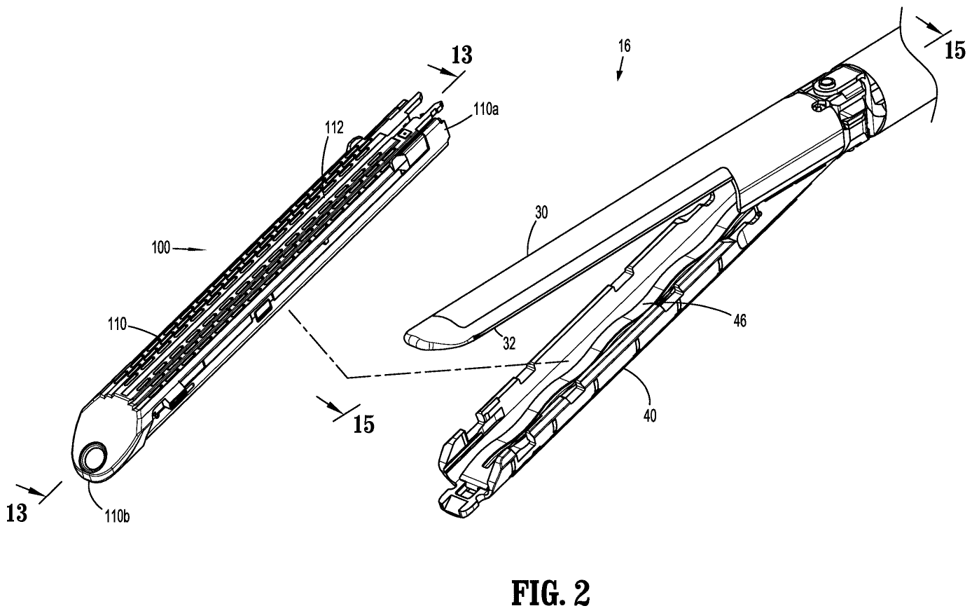

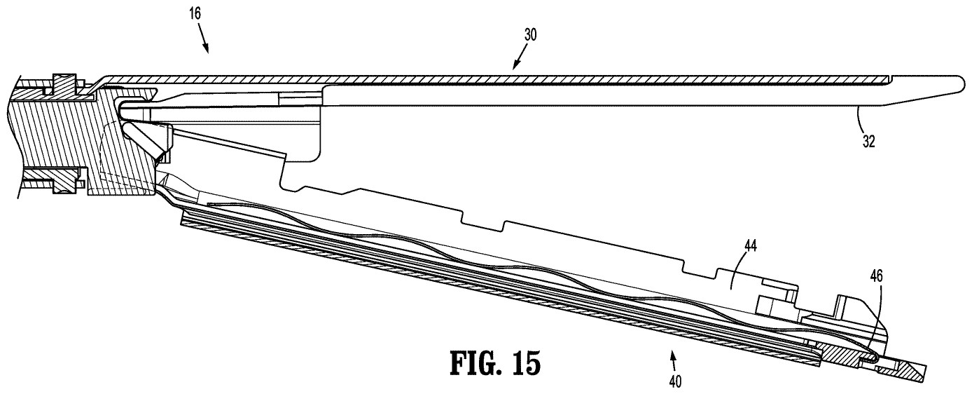

Referring now to FIG. 2, an exploded view of the tool assembly 16 is shown. The tool assembly 16 includes first and second jaws 30, 40 that are pivotable relative to each other between a spaced apart configuration (FIG. 15) and an approximated configuration (FIG. 16). The spaced apart configuration is configured to receive tissue “T” between the first and second jaws 30, 40 while the approximated configuration is configured to grasp tissue “T” between the first and second jaws 30, 40. The tool assembly 16 is shown with a stationary first jaw 30 and a pivotable second jaw 40 and it is envisioned that the arrangement may be reversed providing a pivotable first jaw 30 and a stationary second jaw 40. The first jaw 30 includes an anvil 32 with staple forming pockets (not shown). The second jaw 40 has a U-shaped channel 42 for releasably receiving a staple cartridge 100 that is described in further detail below. The U-shaped channel 42 has a longitudinally extending passage 44 in a bottom surface thereof.

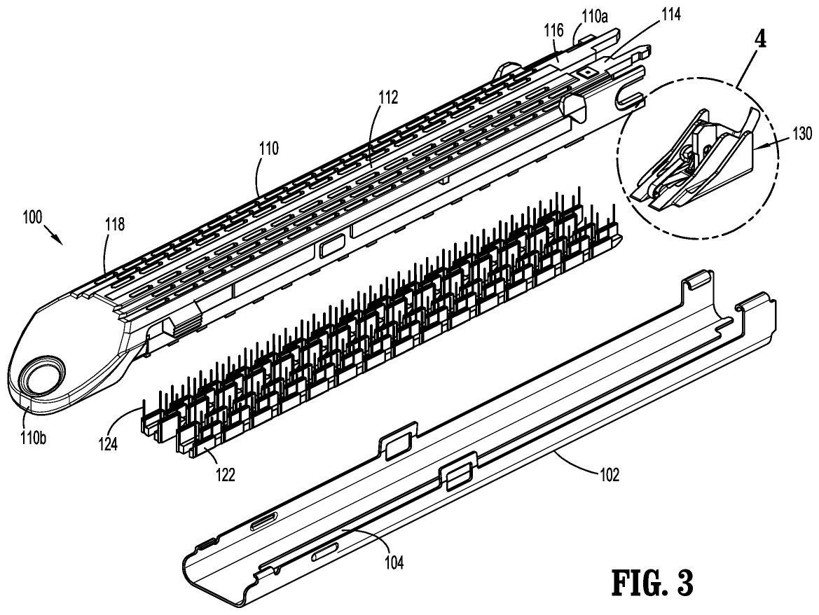

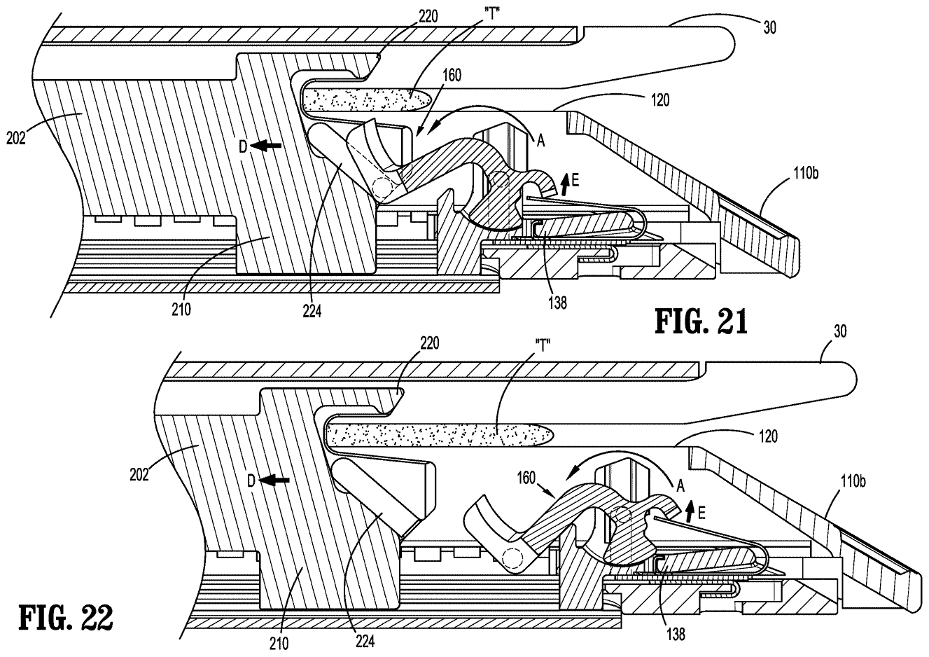

FIG. 3 illustrates an exploded view of the staple cartridge 100. The staple cartridge 100 includes a shield 102 for supporting a cartridge body 110, staple pushers 122 and associated staples 124, and an actuation sled 130. The shield 102 has a slot 104 extending from a proximal end of the shield 102 towards the distal end of the shield 102. The cartridge body 110 has a central slot 112 extending along a tissue contacting surface 120 of the cartridge body 110. The central slot 112 extends along a majority of a length of the cartridge body 110 between proximal and distal portions 110a, 110b of the cartridge body 110. The central slot 112 divides the cartridge body 110 into first and second portions 114, 116. Each of the first and second portions 114, 116 includes longitudinally extending rows of staple receiving pockets 118 where each staple receiving pocket 118 is configured to receive one of the staple pushers 122 and one of the staples 124. The actuation sled 130 is linearly translatable through the cartridge body 110 between a retracted position (FIG. 16) and an advanced position (FIG. 21).

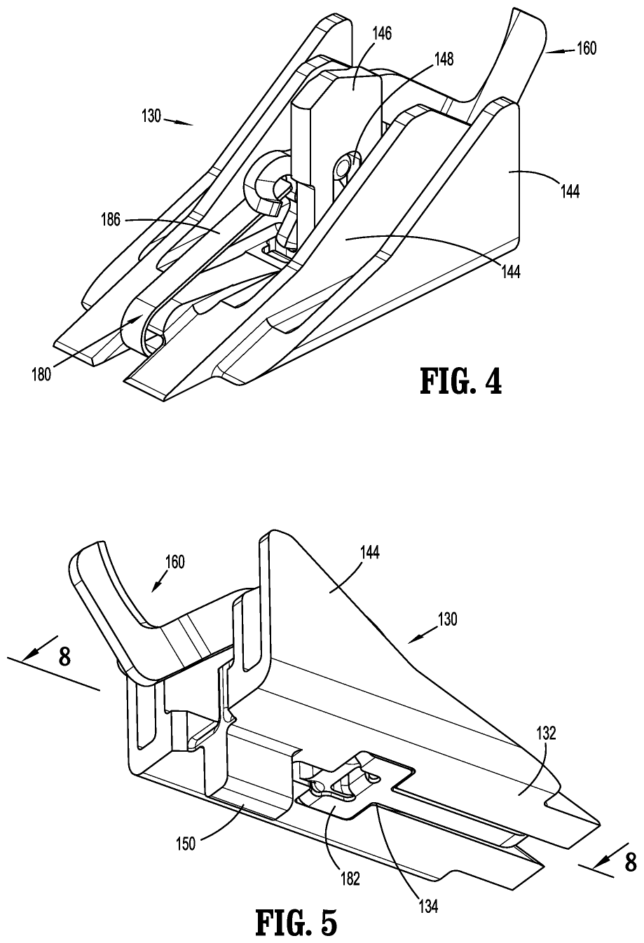

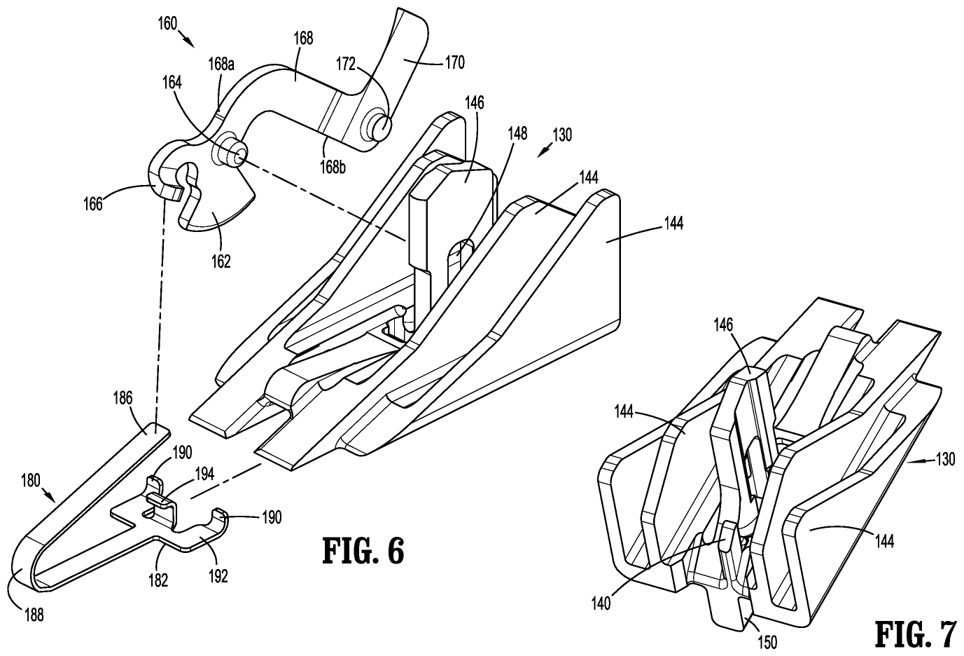

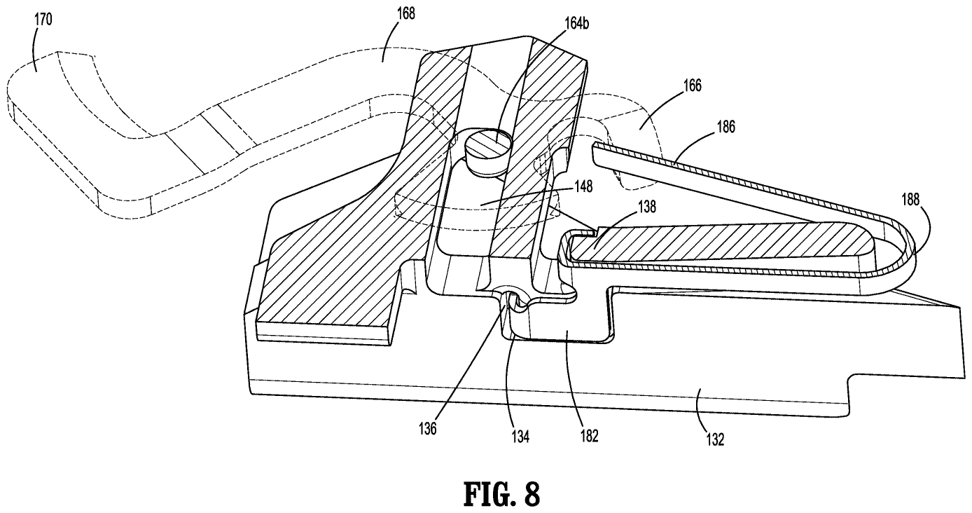

Referring now to FIGS. 4 and 5, the actuation sled 130 has a base 132, angled camming wedges 144, a guide member 146, a knife 160, and a spring 180. The guide member 146 extends vertically and orthogonally from the base 132. The actuation sled 130 may include an optional keel 150 that extends from the base 132 in a direction opposite that of the guide member 146. The keel 150 is configured to stabilize the actuation sled 130 as it translates through the cartridge body 110 as will be explained further hereinafter. The camming wedges 144 are configured to interact with the staple pushers 122 such that linear translation of the actuation sled 130 towards the advanced position urges the staple pushers 122 vertically in the staple receiving pockets 118 thereby ejecting the staples 124 from the staple receiving pockets 118 towards the staple forming pockets (not shown) on the anvil 32. The linear translation of the actuation sled 130 and its interaction with the staple pushers 122 results in sequential ejection of the staples 124 from the staple receiving pockets 118. The guide member 146 has an elongated slot 148 that extends vertically from the base 132 of the actuation sled 130. The knife 160 has a first pin 164 that is slidably and rotatably disposed in the elongated slot 148 of the guide member 146. The spring 180 includes a support 182 disposed in a recess 134 of the base 132 and a finger 186 extending from the support 182 and oriented such that its distal end faces the guide member 146 where it contacts a projection 166 of the knife 160. The finger 186 is resiliently connected to the support 182 by an arcuate portion 188 such that the rest state of the finger 186 defines an acute angle with respect to the support 182. This engagement between the finger 186 of the spring 180 and the projection 166 of the knife 160 biases the knife 160 into the retracted or shielded position of the knife 160. With brief reference to FIGS. 6 and 8, the support 182 includes tabs 190 and a protrusion 194. The tabs 190 are configured to fit into notches 136 in the recess 134 and are disposed on distal ends of tines 192 of the support 182 that define a gap between the tabs 190. The protrusion 194 is located between the tines 192 and is axially spaced from the tabs 190. The protrusion 194 has a C-shaped portion that engages a shelf 138 of the actuation sled 130. The engagement of the protrusion 194 and the shelf 138 in combination with the tabs 190 disposed in the notches 136 of the recess 134 retains the spring 180 in the recess 134 of the actuation sled 130.

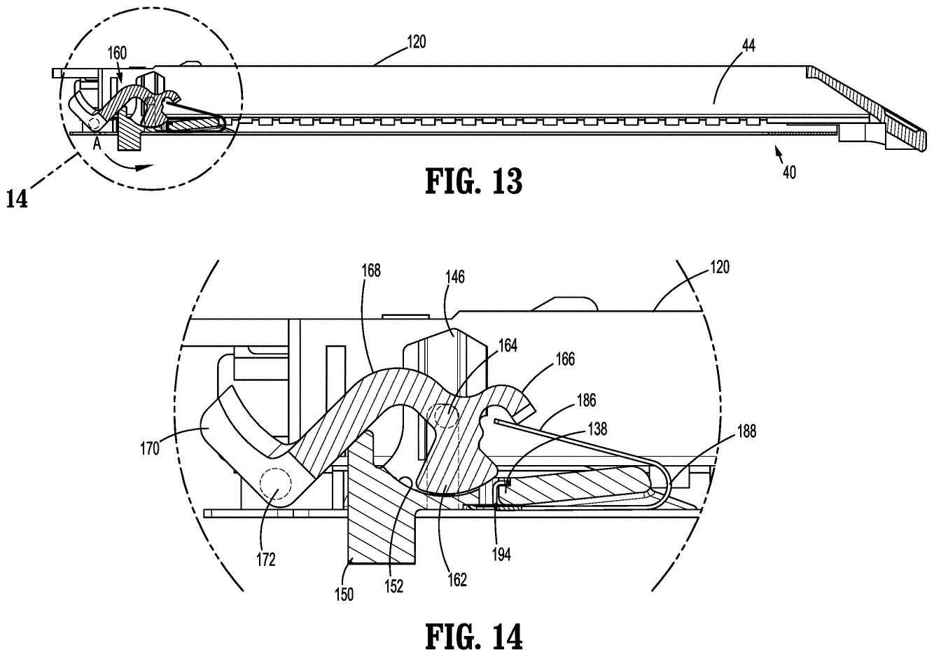

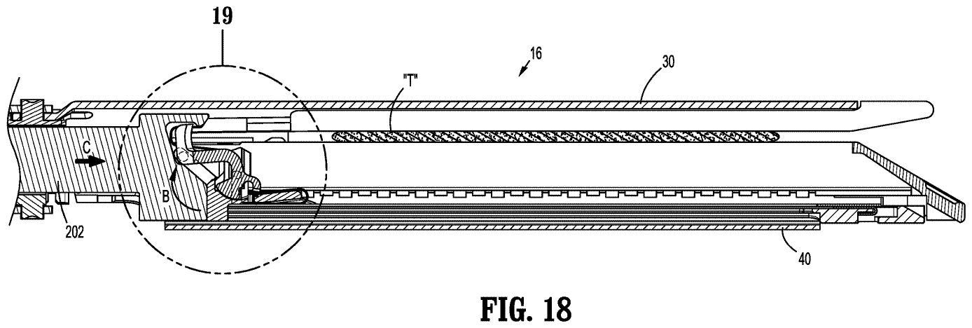

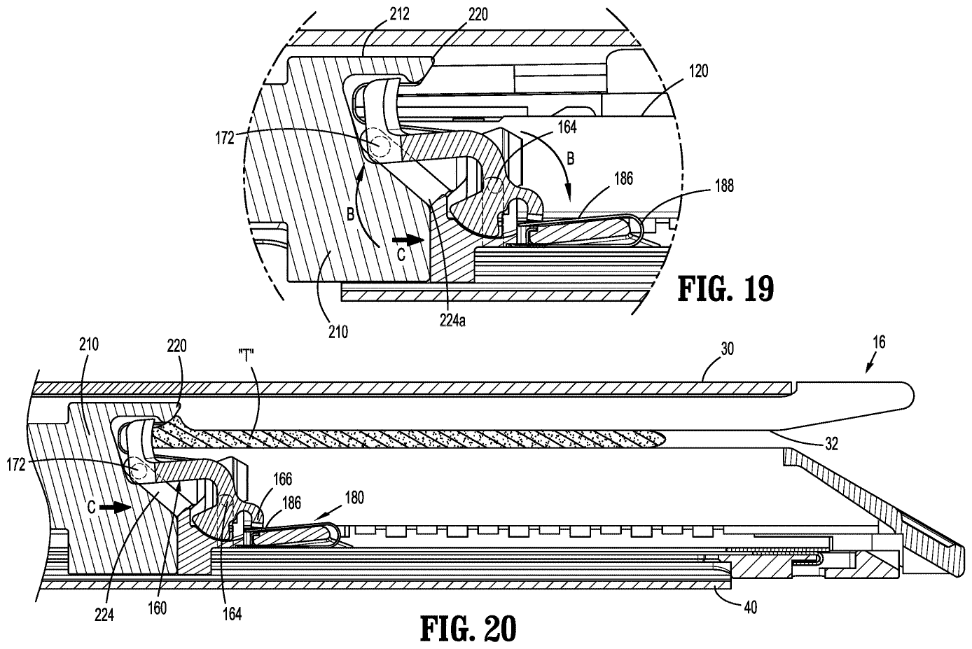

Turning now to FIGS. 6 and 7, the knife 160 includes a foot 162, the projection 166, and an arm 168. The projection 166 extends from the foot 162 in a first direction and the arm 168 extends from the foot 162 in a second direction that is opposed to the first direction. The arm 168 has opposed first and second ends 168a, 168b. The first end 168a of the arm 168 is attached to the foot 162. The projection 166 has a hook shape with an end configured to engage the finger 186 of the spring 180. The first pin 164 is disposed on the foot 162 and extends laterally from the foot 162. A knife blade 170 is attached to the second end 168b of the arm 168 and includes a second pin 172 that extends laterally from the arm 168 in the same direction as the first pin 164. The knife blade 170 extends orthogonally from the arm 168 and is configured to cut tissue “T”. As assembled, the finger 186 of the spring 180 is biased away from the base 132 of the actuation sled 130 and contacts the projection 166. This interaction rotates the knife 160 in a first direction as indicated by arrow “A” (FIG. 13) into the retracted or shielded position (FIG. 13). Rotation of the knife 160 in the shielded position is limited by a position stop 140 located on a proximal end of the actuation sled 130. As the knife 160 rotates in the first direction into the shielded position, a portion of the arm 168 contacts the position stop 140 thereby inhibiting further rotation of the knife 160. In addition to being rotatable in the elongated slot 148 of the guide member 146, the knife 160, via the first pin 164, is also vertically repositionable in the elongated slot 148. In particular, as the knife 160 rotates in the direction indicated by arrow “B” (FIGS. 18 and 19), the foot 162 engages an arcuate cut surface 152 that urges the first pin 164 vertically in the elongated slot 148 setting a vertical position of the knife 160. The elongated slot 148 is dimensioned such that the first pin 164 is capable of limited proximal and distal movement in the elongated slot 148 in addition to being rotatable and vertically repositionable in the elongated slot 148 (FIG. 8).

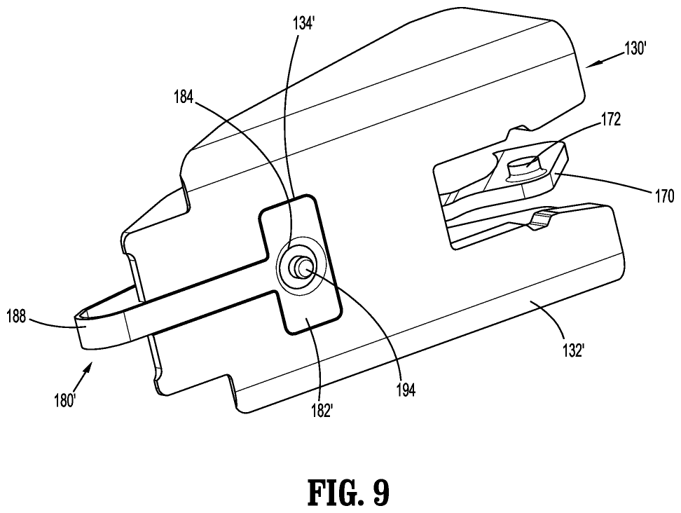

With brief reference to FIG. 9, another aspect of the actuation sled 130′, recess 134′, and spring 180′ is shown. Rather than using the tabs 190 and protrusion 194 to attach the spring 180′ to the actuation sled 130′, the support 182′ has a rectangular base 132′ with a central opening 184 such that the spring 180′ can be attached to the base 132′ of the actuation sled 130′ by heat staking the base 132′ of the support 182′ to a post 142 on the base 132′ of the actuation sled 130′.

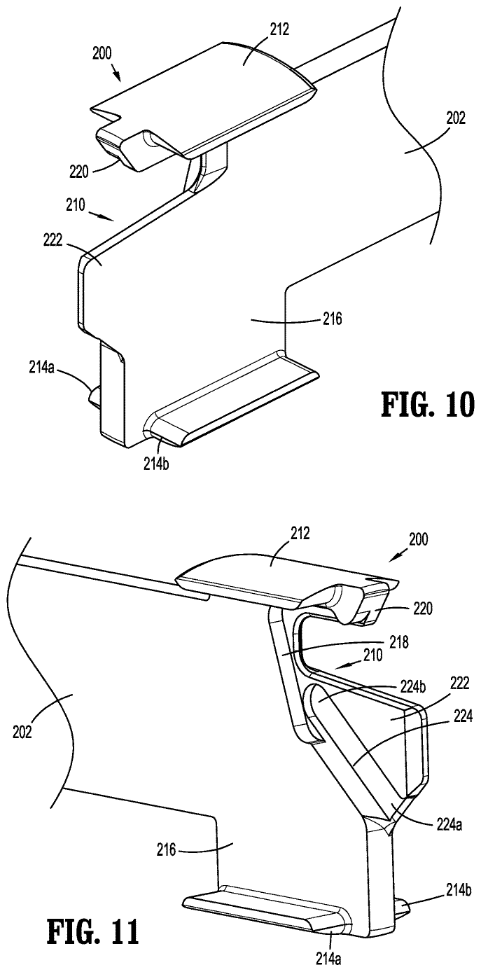

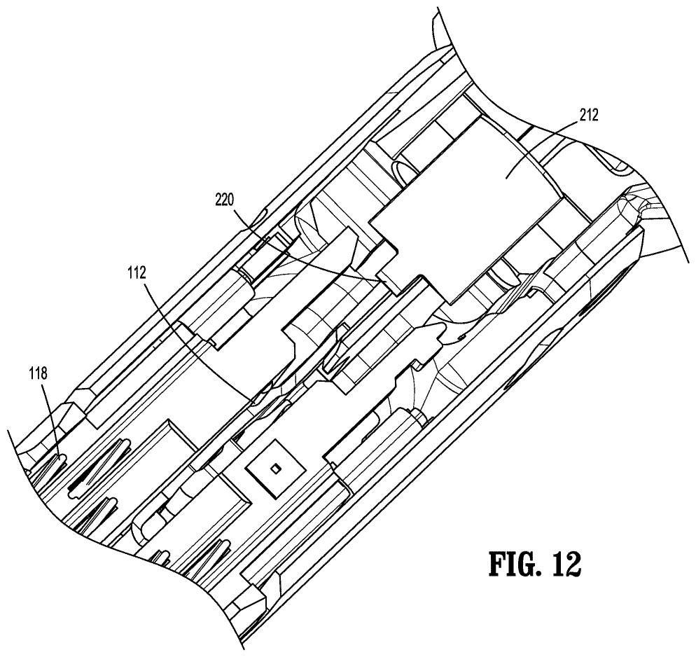

Turning now to FIGS. 10 and 11, a distal portion of a drive member 200 of the stapling device 10 is shown. The drive member 200 is linearly translatable through the shaft 14 between a retracted position (FIGS. 12 and 16) and an advanced position (FIG. 21). The drive member 200 includes an elongated beam 202 that terminates in a working member 210. It is contemplated that the elongated beam 202 may be formed from multiple layers or laminates as well as multiple beams. In aspects of the disclosure, the working member 210 is welded to the beam 202. Alternatively, the working member 210 can be secured to the beam 202 using a variety of different securement techniques or devices. The working member 210 has an I-beam configuration and includes a first plate 212, first and second wings 214a, 214b, and a vertical strut 216 that connects the first plate 212 to the first and second wings 214a, 214b. The opposed wings 214a, 214b extend laterally from the vertical strut 216. The vertical strut 216 has a distally facing cam surface 218. The first plate 212 also supports, or is formed with, a nose 220 that extends at an acute angle from the first plate 212 towards the first and second wings 214a, 214b. The nose 220 is configured to bluntly direct tissue “T” towards the first and second wings 214a, 214b (FIG. 20). An appendix 222 extends longitudinally from the vertical strut 216 and includes a groove 224. The groove 224 is angled with respect to the vertical strut 216 and has an open distal end 224a and a closed proximal end 224b. The groove 224 is configured to slidably receive the second pin 172 of the knife 160 as will be described in detail hereinafter.

As shown in FIG. 12, the drive member 200 is in the retracted position and the knife 160 is in the shielded position. The guide member 146 and the knife blade 170 are aligned with the central slot 112 of the cartridge body. Thus, during translation of the actuation sled 130 and the drive member 200, the guide member 146 and the knife blade 170 translate along the central slot 112 of the cartridge body 110.

Referring now to FIGS. 13 and 14, a side cross-sectional view of the cartridge body 110 is shown with the actuation sled 130 in a retracted position that is defined by the actuation sled 130 being located at the proximal portion 110a of the cartridge body 110. In the retracted position, the knife 160 is in the shielded position due to its rotation in the first direction as indicated by arrow “A” that is a result of the finger 186 of the spring 180 acting on a portion of the projection 166 of the knife 160 that rotates the knife 160 about the first pin 164 located in the elongated slot 148 of the guide member 146. Rotation in the first direction is limited by engagement of a portion of the arm 168 of the knife 160 with the position stop 140 on the actuation sled 130. In the shielded position, the knife blade 170 is positioned below the tissue contacting surface 120 of the cartridge body 110 such that it does not contact tissue “T” disposed on the tissue contacting surface 120 of the cartridge body 110 (FIG. 16). Additionally, when used, the optional keel 150 of the actuation sled 130 extends through the slot 104 of the shield 102 and assists in guiding the actuation sled 130 during linear translation through the cartridge body 110 and improving lateral stability of the actuation sled 130 during linear translation.

With reference now to FIGS. 2 and 15, the first jaw 30 also includes a wave spring 46 position between a bottom surface of the U-shaped channel 42 and a bottom surface of the cartridge body 110. The wave spring 46 is compressible and applies a biasing force to the cartridge body 110 of the cartridge assembly 100. When the first and second jaws 30, 40 are in the approximated position with tissue “T” disposed between the anvil 32 and the tissue contacting surface 120 of the cartridge body 110 (FIG. 16), the wave spring 46 allows vertical movement of the cartridge body 110 of the cartridge assembly 100 in the U-shaped channel 42 to account for different tissue thicknesses. Thus, thicker tissue between the anvil 32 and the cartridge body 110 compresses the wave spring 46 more than thinner tissue thereby applying a substantially constant or uniform pressure on tissue captured between the anvil 32 and the cartridge body 110.

Referring now to FIGS. 16-22, operation of the tool assembly 16 is illustrated. The tool assembly 16 is shown in the approximated configuration with tissue “T” captured between the anvil 32 and the tissue contacting surface 120 of the cartridge body 110. Initially, the drive member 200 and the actuation sled 130 are in their retracted positions and the knife 160 is in the shielded position. Further, the second pin 172 of the knife 160 is slidably disposed at the open distal end 224a of the groove 224 of the working member 210. During actuation of the stapling device 10, the drive member 200 translates linearly through the tool assembly 16 in a distal direction towards the advanced position. As the drive member 200 and the working member 210 translate distally through the tool assembly 16, the second pin 172 slides in the groove 224 of the working member 210 from the open distal end 224a towards the closed proximal end 224b. Sliding the second pin 172 from the open distal end 224a of the groove 224 to the closed proximal end 224b of the groove 224 overcomes the bias applied by the finger 186 of the spring 180 thereby rotating the knife 160 in the direction indicated by arrow “B” (FIGS. 18 and 19) into the exposed or cutting position. In the cutting position, the knife blade 170 is exposed and adapted to cut tissue “T” positioned between the anvil 32 and the tissue contacting surface 120 of the cartridge body 110. When the knife 160 is rotated into the cutting position, the knife blade 170 contacts the cam surface 218 and positions the knife blade 170 for cutting tissue “T”. In some aspects of the disclosure, in the cutting position, the knife blade 170 (FIG. 20) is angled in relation to an axis that is transverse to a longitudinal axis of the tool assembly (not perpendicular) to perform a slicing action which promotes more efficient cutting of the tissue “T”. The amount of contact between the knife blade 170 and the cam surface 218 of the drive member 200 varies according to the amount of compression applied to the wave spring 46. When the cartridge assembly 100 is down relative to a bottom surface of the shield 102, this compresses the wave spring 46 and contact between the knife blade 170 and the cam surface 218 is at a minimum. Conversely, when the cartridge assembly 100 is up relative to the bottom surface of the shield 102, this allows the wave spring 46 to become uncompressed and the knife blade 170 rests against the cam surface 218 thereby supporting the knife blade 170. As seen in FIG. 18, the first and second jaws 30, 40 of the tool assembly 16 are in the approximated configuration with tissue “T” captured therebetween and the drive member 200 is translating in the direction indicated by arrow “C” such that the knife 160 is rotated into the cutting position as the actuation sled 130 translates distally along with the drive member 200. A distal end of the working member 210 contacts a proximal end of the actuation sled 130 and urges the actuation sled 130 distally through the cartridge body 110. During distal translation of the working member 210 and actuation sled 130, tissue “T” disposed between the first and second jaws 30, 40 of the tool assembly 16 contacts the nose 220 of the working member 210 and is directed towards the knife blade 170 thereby improving the cutting of tissue “T” by the knife blade 170. After the drive member 200 and the actuation sled 130 reach their advanced positions (FIG. 21), the drive member 200 starts proximal translation through the cartridge body 110 towards the retracted position as indicated by arrow “D” while the actuation sled 130 remains in the advanced position in the distal portion 110b of the cartridge body 110. As the drive member 200 translates towards the retracted position (FIG. 16), the second pin 172 slides from the closed proximal end 224b of the groove 224 to the open distal end 224a of the groove 224 thereby rotating the knife 160 in the first direction as indicated by arrow “A” into the shielded position (FIG. 22). The finger 186 moves in the direction indicated by arrow “E” such that the finger 186 contacts the projection 166 of the knife 160 thereby maintaining the knife 160 in the shielded position due to the biasing force of the finger 186 being applied in the direction indicated by arrow “E”. With the knife 160 in the shielded position, the cartridge body 110 can be safely removed from the U-shaped channel 42 of the second jaw 40. Additionally, since the actuation sled 130 and knife 160 remain in the distal portion 110b of the cartridge body 110 after an actuation sequence, the cartridge body 110 and the knife 160 are only used once before being discarded. Additionally, if a partial actuation sequence is used, proximal movement of the drive member 200 separates the drive member 200 from the actuation sled 130 as previously described. Thus, whether a partial actuation or a full actuation is effected, proximal movement of the drive member 200 separates the drive member 200 from the actuation sled 130 thereby translating the knife 160 into the shielded position as described hereinabove.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A staple cartridge comprising:
a cartridge body having a central slot extending along a length of the cartridge body;
an actuation sled including a body having a base, the body having:
a guide member including a slot, the guide member and slot extending from the base in an orientation that is transverse to the base,
angled camming wedges disposed on opposed sides of the guide member and a recess located in the base, and
a spring including a support and a finger resiliently coupled to the support; and
a knife including a foot, an arm, and a projection, the arm and the projection extending in opposite directions from the foot, the foot having a first pin disposed in the slot such that the pin is slidable in the slot and the knife is rotatable relative to the guide member, the arm including a second pin and a knife blade, the second pin engageable with a groove of a drive member, and the projection engaging the finger such that the finger biases the projection in a first direction thereby urging the arm in the first direction and defining a shielded position of the knife.

2. The staple cartridge of claim 1, wherein translation of the drive member from a retracted position towards an advanced position causes the second pin to slide from a distal position of the groove towards a proximal position of the groove thereby rotating the knife in a second direction opposite the first direction.

3. The staple cartridge of claim 2, wherein translation of the drive member towards the advanced position overcomes the bias of the finger such that the knife rotates in the second direction towards a cutting position in which the knife blade is angled in relation to an axis that is transverse to a longitudinal axis of the staple cartridge.

4. The staple cartridge of claim 1, wherein the drive member includes a nose that defines an acute angle with respect to a longitudinal axis of the staple cartridge and is configured to urge tissue towards the knife blade.

5. The staple cartridge of claim 1, wherein the cartridge body further includes staples disposed in a corresponding number of staple receiving pockets.

6. The staple cartridge of claim 5, further including pushers positioned within the staple receiving pockets and supporting the staples, wherein the angled camming wedges are movable into engagement with the pushers to eject the staples from the staple receiving pockets.

7. The staple cartridge of claim 2, wherein translation of the drive member from the advanced position towards the retracted position causes the second pin to slide in the groove from the proximal position towards the distal position.

8. The staple cartridge of claim 7, wherein translation of the drive member from the advanced position towards the retracted position separates the actuation sled from the drive member thereby rotating the knife in the first direction into the shielded position.

9. A surgical stapling instrument comprising:
a handle;
a shaft extending from the handle;
a reload assembly coupled to the shaft, the reload assembly including a drive member and a working member, the drive member translatable between retracted and advanced positions, the working member disposed at a distal end of the drive member; and
a tool assembly coupled to the reload assembly, the tool assembly including an anvil and a cartridge assembly that are pivotable with respect to each other between open and clamped configurations, the cartridge assembly including a receptacle and a staple cartridge disposed in the receptacle, the staple cartridge having:
a cartridge body including a central slot,
an actuation sled including a body and a base,
a guide member including an elongated slot, the guide member and the elongated slot extending transversely to the base,
a spring having a support and a finger extending resiliently from the support, the support disposed in a recess of the base, and
a knife having a foot, an arm, and a projection, the arm and projection extending in opposite directions from the foot, the foot including a first pin disposed in the slot such that the knife is rotatable relative to the guide member, the arm including a knife blade and a second pin, the second pin engageable with a groove in the working member, the projection engaging the finger such that the finger biases the projection in a first direction thereby urging the arm in the first direction and defining a shielded position of the knife, the knife transitionable to a cutting position as the drive member translates from the retracted position towards the advanced position.

10. The surgical stapling instrument of claim 9, wherein translation of the drive member from the retracted position towards the advanced position causes the second pin to slide from a distal position of the groove towards a proximal position of the groove thereby rotating the knife in a second direction opposite the first direction.

11. The surgical stapling instrument of claim 10, wherein translation of the drive member towards the advanced position overcomes the bias of the finger such that the knife rotates in the second direction towards an exposed position.

12. The surgical stapling instrument of claim 9, wherein the working member includes a nose that defines an acute angle with respect to a longitudinal axis of the staple cartridge and is configured to urge tissue towards the knife blade.

13. The surgical stapling instrument of claim 9, wherein the cartridge body further includes staples disposed in a corresponding number of staple receiving pockets.

14. The surgical stapling instrument of claim 13, further including pushers positioned within the staple receiving pockets and supporting the staples, wherein the angled wedges are movable into engagement with the pushers to eject the staples from the staple receiving pockets.

15. The surgical stapling instrument of claim 10, wherein translation of the drive member towards the retracted position causes the second pin to slide in the groove from the proximal position towards the distal position.

16. The surgical stapling instrument of claim 15, wherein translation of the drive member towards the retracted position separates the actuation sled from the working member thereby rotating the knife in the first direction into the shielded position.

17. A tool assembly for use with a surgical stapling instrument, the tool assembly comprising:
an anvil; and
a cartridge assembly, the anvil and cartridge assembly being pivotable relative to each other between open and clamped positions, the cartridge assembly including:
a receptacle,
a cartridge body including a central slot,
an actuation sled translatable in the cartridge body, the actuation sled including a base having a recess,
a guide member extending from the base, the guide member including an elongated slot that is transverse to the base,
a spring coupled to the base, the spring including a resilient finger, and
a knife having a foot, an arm, and a projection, the foot including a first pin positioned in the elongated slot such that the knife is pivotable relative to the guide member, the arm including a knife blade and a second pin, the second pin slidably engageable with a groove of a drive member, and the projection operatively coupled with the resilient finger such that the resilient finger biases the projection in a first direction and defining a shielded position of the knife, the knife transitionable to a cutting position as the drive member translates linearly through the cartridge body from a retracted position towards an advanced position wherein the second pin slides in the groove at an acute angle with respect to a longitudinal axis of the cartridge body.

18. The tool assembly of claim 17, wherein translation of the drive member from the retracted position towards the advanced position causes the second pin to slide from a distal position of the groove towards a proximal position of the groove thereby rotating the knife in a second direction opposite the first direction.

19. The tool assembly of claim 17, wherein the drive member includes a nose that defines an acute angle with respect to the longitudinal axis and is configured to urge tissue towards the knife blade.

20. The tool assembly of claim 17, wherein translation of the drive member from the advanced position towards the retracted position causes the second pin to slide in the groove from the proximal position towards the distal position and separates the actuation sled from the drive member, the finger biasing the projection in the first direction thereby rotating the knife in the first direction into the shielded position.
